(19) United States
(12) Patent Application Publication
(10) Pub. No.: US 2016/0038059 A1
(43) Pub. Date: Feb. 11, 2016

(54) GAIT POSTURE METER AND PROGRAM

(71) Applicant: OMRON HEALTHCARE CO., LTD., Muko-shi (JP)

(72) Inventors: Yuji ASADA, Kyoto (JP); Naoki TAKEISHI, Kyoto (JP); Yumi KITAMURA, Kyoto (JP)

(21) Appl. No.: 14/918,959

(22) Filed: Oct. 21, 2015

Related U.S. Application Data
(63) Continuation of application No. PCT/JP2014/058916, filed on Mar. 27, 2014.

(30) Foreign Application Priority Data
May 10, 2013 (JP) 2013-100625

(51) Int. Cl. A61B 5/11

(52) U.S. Cl. CPC A61B 5/112; A61B 5/1116

(57) ABSTRACT

A gait posture meter according to the invention includes an accelerometer affixed to a centerline of a measurement subject's waist area, and physical amounts corresponding to the measurement subject's waist position while walking are quantitatively calculated using one or both of an up-down axis acceleration timewise change waveform and a front-rear axis acceleration timewise change waveform outputted by the accelerometer.

### BACKGROUND OF THE INVENTION

1. Field of the Invention

This invention relates to gait posture meters, and particularly relates to a gait posture meter that quantitatively evaluates whether or not a person's gait posture is a correct posture.

This invention also relates to a program for causing a computer to execute a method that quantitatively evaluates whether or not a person's gait posture is a correct posture.

2. Description of the Related Art

The apparatus disclosed in JP 2011-078728A, for example, detects a gravity acceleration direction based on an output from an accelerometer affixed to the waist area of a person when the person takes on a predetermined posture, finds an angle of tilt of the waist area relative to the ground when that posture is taken, and from this angle of tilt estimates a tilt in the pelvis.

The device disclosed in JP 2011-251013A calculates a movement amount from an output of an accelerometer affixed to the waist area of a person and obtains a walking trajectory based on that movement amount.

### SUMMARY OF THE INVENTION

Incidentally, supporting the pelvis on the leg that has been put forward, or in other words, having the waist shifted forward relative to the rest of the body, is an important factor for a person to walk with a correct posture.

However, there has thus far not been a means for easily evaluating whether or not the waist position is shifted forward during walking in a quantitative manner. As such, users have relied on sensory evaluations when evaluating whether or not a gait posture is a correct posture, which has been inconvenient when, for example, undergoing training to correct the gait posture.

Accordingly, this invention provides a gait posture meter capable of easily evaluating whether or not the waist position is shifted forward during walking in a quantitative manner.

In addition, this invention also provides a program for causing a computer to execute a method capable of easily evaluating whether or not the waist position is shifted forward during walking in a quantitative manner.

To solve the aforementioned problems, a gait posture meter according to this invention is a gait posture meter that evaluates a gait posture of a measurement subject, including an accelerometer affixed to a centerline of a measurement subject's waist area, a computation unit that quantitatively calculates a physical amount corresponding to a waist position of the measurement subject while walking in a front-rear direction relative to the rest of the body using one or both of a timewise change waveform of an up-down axis acceleration and a timewise change waveform of a front-rear axis acceleration outputted by the accelerometer, and an evaluation unit that evaluates whether or not the waist position while walking is shifted forward in the front-rear direction relative to the rest of the body based on the physical amount.

In the present specification, “waist position” refers to the position of the waist relative to the rest of the body while walking. Typically, this is defined as follows, using a stride length (that is, a distance from the tip of the toe of the rear foot to the heel of the front foot) and a distance from a rear surface of the waist to the heel of the front foot, at the point in time when the heel of the front foot makes contact with the ground.

(waist position)=(distance from rear surface of waist to heel of front foot)/(stride length)

In the gait posture meter according to this invention, the accelerometer is affixed to the centerline of the measurement subject's waist area. The computation unit quantitatively calculates a physical amount corresponding to the waist position of the measurement subject while walking in a front-rear direction relative to the rest of the body using one or both of the timewise change waveform of the up-down axis acceleration and the timewise change waveform of the front-rear axis acceleration outputted by the accelerometer. The evaluation unit evaluates whether or not the waist position while walking is shifted forward in the front-rear direction relative to the rest of the body based on the physical amount. Accordingly, whether or not the measurement subject's waist position while walking is shifted forward can be quantitatively evaluated. Furthermore, the gait posture meter carries out the evaluation based on the output of the stated accelerometer, and thus the evaluation can be made easily, without requiring large-scale equipment such as is used for motion capture or the like.

In a gait posture meter according to a preferred embodiment, the computation unit includes a signal processing system that combines the up-down axis acceleration and the front-rear axis acceleration, and the physical amount includes an amount regarding a combined vector obtained by combining the up-down axis acceleration and the front-rear axis acceleration.

In the case where the waist position is shifted forward, it is known from experience that when the rear leg, which serves as a reference, is kicked out, accelerations occur simultaneously in both the upward and forward directions. Conversely, in the case where the waist position is not shifted forward, it is known from experience that when the rear leg serving as a reference is kicked out, an acceleration first occurs in the upward direction in order to lift the body, after which an acceleration occurs in the forward direction. In this manner, whether or not the waist position is shifted forward is related to both the up-down axis acceleration and the front-rear axis acceleration. Here, in the gait posture meter according to this preferred embodiment, the computation unit includes the signal processing system that combines the up-down axis acceleration and the front-rear axis acceleration, and the physical amount includes an amount regarding the combined vector obtained by combining the up-down axis acceleration and the front-rear axis acceleration. Accordingly, whether or not the waist position is shifted forward can be correctly evaluated in accordance with an amount regarding the stated combined vector.

In a gait posture meter according to a preferred embodiment, the amount regarding the combined vector is a magnitude of the combined vector.

In the case where the waist position is shifted forward, accelerations occur simultaneously in both the upward and forward directions when the rear leg serving as a reference is kicked out, and thus a peak where the combined vector is high appears in each of a left leg reference period spanning from when a left heel makes contact with the ground to when a right heel makes contact with the ground and a right leg reference period spanning from when the right heel makes contact with the ground to when the left heel makes contact with the ground. Conversely, in the case where the waist position is not shifted forward, when the rear leg serving as a reference is kicked out, an acceleration first occurs in the upward direction in order to lift the body, after which an acceleration occurs in the forward direction; accordingly, the peak in the combined vector is smaller in both the left leg reference period spanning from when the left heel makes contact with the ground to when the right heel makes contact with the ground and the right leg reference period spanning from when the right heel makes contact with the ground to when the left heel makes contact with the ground. Here, in the gait posture meter according to this preferred embodiment, the amount regarding the combined vector is the magnitude of the combined vector. Accordingly, whether or not the waist position is shifted forward can be correctly evaluated in accordance with the magnitude of the stated combined vector.

In a gait posture meter according to a preferred embodiment, the physical amount includes an amount expressing a positive-side waveform area and/or a negative-side waveform area in the timewise change waveform of the up-down axis acceleration, in each of the left leg reference period spanning from when the left heel makes contact with the ground to when the right heel makes contact with the ground and a right leg reference period spanning from when the right heel makes contact with the ground to when the left heel makes contact with the ground.

In the present specification, “positive-side waveform area” refers to an area obtained by integrating, with respect to time, a waveform in a time-acceleration graph when the acceleration has a positive value. Likewise, “negative-side waveform area” refers to an area obtained by integrating, with respect to time, a waveform in a time-acceleration graph when the acceleration has a negative value.

In the case where the waist position is shifted forward, the stride length is long and the walking speed is high; as such, it is known from experience that in both the left leg reference period and the right leg reference period in the timewise change waveform of the up-down axis acceleration, the positive-side waveform area and/or the negative-side waveform area become greater. Conversely, in the case where the waist position is not shifted forward, the stride length is short and the walking speed is low; as such, it is known from experience that in both the left leg reference period and the right leg reference period in the timewise change waveform of the up-down axis acceleration, the positive-side waveform area and/or the negative-side waveform area become smaller. In this manner, whether or not the waist position is shifted forward is related to the positive-side waveform area and/or the negative-side waveform area in the stated timewise change waveform of the up-down axis acceleration. Here, in the gait posture meter according to this preferred embodiment, the physical amount includes an amount expressing the positive-side waveform area and/or the negative-side waveform area in the timewise change waveform of the up-down axis acceleration, in each of the left leg reference period and the right leg reference period. Accordingly, whether or not the waist position is shifted forward can be evaluated correctly in accordance with the amount expressing the positive-side waveform area and/or the negative-side waveform area in the stated timewise change waveform of the up-down axis acceleration.

In a gait posture meter according to a preferred embodiment, the physical amount includes an amount expressing a value of a lowest valley in the negative-side waveform in the timewise change waveform of the up-down axis acceleration, in each of the left leg reference period spanning from when the left heel makes contact with the ground to when the right heel makes contact with the ground and the right leg reference period spanning from when the right heel makes contact with the ground to when the left heel makes contact with the ground.

In the case where the waist position is shifted forward, the stride length is long and the walking speed is high; as such, it is known from experience that in both the left leg reference period and the right leg reference period in the timewise change waveform of the up-down axis acceleration, a lowest valley in the negative-side waveform becomes deeper. Conversely, in the case where the waist position is not shifted forward, the stride length is short and the walking speed is low; as such, it is known from experience that in both the left leg reference period and the right leg reference period in the timewise change waveform of the up-down axis acceleration, the lowest valley in the negative-side waveform becomes shallower. In this manner, whether or not the waist position is shifted forward is related to the depth of the lowest valley in the negative-side waveform in stated timewise change waveform of the up-down axis acceleration. Here, in the gait posture meter according to this preferred embodiment, the physical amount includes an amount expressing the value of the lowest valley in the negative-side waveform in the timewise change waveform of the up-down axis acceleration, in each of the left leg reference period and the right leg reference period. Accordingly, whether or not the waist position is shifted forward can be evaluated correctly in accordance with the amount expressing the value of the lowest valley in the negative-side waveform in the stated timewise change waveform of the up-down axis acceleration.

Note that the magnitude of a highest peak in the positive-side waveform for the timewise change waveform of the up-down axis acceleration corresponds less to the waist position and rather varies greatly from person to person, and is therefore difficult to use for a quantitative evaluation.

In a gait posture meter according to a preferred embodiment, the physical amount includes an amount expressing a value of a highest peak in a positive-side waveform and/or a value of a lowest valley in a negative-side waveform in the timewise change waveform of the front-rear axis acceleration, in each of the left leg reference period spanning from when the left heel makes contact with the ground to when the right heel makes contact with the ground and the right leg reference period spanning from when the right heel makes contact with the ground to when the left heel makes contact with the ground.

In the case where the waist position is shifted forward, the stride length is long and the walking speed is high; as such, it is known from experience that in both the left leg reference period and the right leg reference period in the timewise change waveform of the front-rear axis acceleration, the highest peak in the positive-side waveform becomes higher while the lowest valley in the negative-side waveform becomes deeper. Conversely, in the case where the waist position is not shifted forward, the stride length is short and the walking speed is low; as such, it is known from experience that in both the left leg reference period and the right leg reference period in the timewise change waveform of the front-rear axis acceleration, the highest peak in the positive-side waveform becomes lower while the lowest valley in the negative-side waveform becomes shallower. In this manner, whether or not the waist position is shifted forward is related to the height of the highest peak in the positive-side waveform and the depth of the lowest valley in the negative-side waveform in the stated timewise change waveform of the front-rear axis acceleration. Here, in the gait posture meter according to this preferred embodiment, the physical amount includes an amount expressing the value of the highest peak in the positive-side waveform and/or the value of the lowest valley in the negative-side waveform in the timewise change waveform of the front-rear axis acceleration, in each of the left leg reference period spanning from when the left heel makes contact with the ground to when the right heel makes contact with the ground and the right leg reference period spanning from when the right heel makes contact with the ground to when the left heel makes contact with the ground. Accordingly, whether or not the waist position is shifted forward can be evaluated correctly in accordance with the amount expressing the value of the highest peak in the positive-side waveform and/or the value of the lowest valley in the negative-side waveform in the stated timewise change waveform of the front-rear axis acceleration.

In a gait posture meter according to a preferred embodiment, the evaluation unit sets a threshold for the physical amount and evaluates the waist position while walking in the front-rear direction relative to the rest of the body across multiple stages in accordance with the threshold.

In the gait posture meter according to this preferred embodiment, the evaluation unit sets a threshold for the physical amount and evaluates the waist position while walking in the front-rear direction relative to the rest of the body across multiple stages in accordance with the threshold. Accordingly, evaluation results are obtained across multiple stages. Such evaluation results across multiple stages are easy to understand and simple for users (including measurement subjects).

A program according to this invention is a program for causing a computer to execute a method for evaluating a gait posture of a measurement subject, the method including a step of obtaining an output of an accelerometer that is affixed to a centerline of the measurement subject's waist area, a step of quantitatively calculating a physical amount corresponding to a waist position of the measurement subject while walking in a front-rear direction relative to the rest of the body using one or both of a timewise change waveform of a front-rear axis acceleration and a timewise change waveform of an up-down axis acceleration outputted by the accelerometer, and a step of evaluating whether or not the waist position while walking is shifted forward in the front-rear direction relative to the rest of the body based on the physical amount.

By causing a computer to execute the program according to this invention, the computer first obtains the output of the accelerometer that is affixed to the centerline of the measurement subject's waist area. Then, a physical amount corresponding to a waist position of the measurement subject while walking in a front-rear direction relative to the rest of the body is quantitatively calculated using one or both of the timewise change waveform of the front-rear axis acceleration and the timewise change waveform of the up-down axis acceleration outputted by the accelerometer. Furthermore, whether or not the waist position while walking is shifted forward in the front-rear direction relative to the rest of the body is evaluated based on the physical amount. Accordingly, whether or not the measurement subject's waist position while walking is shifted forward can be quantitatively evaluated. Furthermore, the program carries out the evaluation based on the output of the stated accelerometer, and thus the evaluation can be made easily, without requiring large-scale equipment such as is used for motion capture or the like.

As is clear from the foregoing, according to the gait posture meter of this invention, whether or not the waist position is shifted forward during walking can be evaluated easily in a quantitative manner.

In addition, by causing a computer to execute the program according to this invention, whether or not the waist position is shifted forward during walking can be evaluated easily in a quantitative manner.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a diagram illustrating a system configuration of a gait posture meter according to a preferred embodiment of this invention.

FIG. 2 is a diagram illustrating a block configuration of an activity meter that forms part of the system of the stated gait posture meter.

FIG. 3 is a diagram illustrating a block configuration of a smartphone that forms part of the system of the stated gait posture meter.

FIG. 4A is a diagram illustrating the activity meter being affixed to a measurement subject. FIG. 4B is a diagram illustrating an X axis (a front-rear axis), a Y axis (a left-right axis), and a Z axis (an up-down axis).

FIGS. 5A and 5B are diagrams illustrating definitions of waist positions relative to the rest of the body.

FIG. 6 is a diagram illustrating a timewise change waveform of an up-down axis acceleration and a front-rear axis acceleration outputted by an accelerometer for a given measurement subject.

FIG. 7 is a diagram illustrating a timewise change waveform of a combined acceleration obtained by combining the up-down axis acceleration and the front-rear axis acceleration in FIG. 6.

FIG. 8 is a diagram illustrating a timewise change waveform of an up-down axis acceleration and a front-rear axis acceleration outputted by an accelerometer for a different measurement subject from the measurement subject illustrated in FIG. 6.

FIG. 9 is a diagram illustrating a timewise change waveform of a combined acceleration obtained by combining the up-down axis acceleration and the front-rear axis acceleration in FIG. 8.

FIG. 10 is a diagram illustrating a flow of operations performed by a control unit of the stated activity meter.

### DETAILED DESCRIPTION OF THE PREFERRED EMBODIMENTS

Hereinafter, a preferred embodiment of the invention will be described in detail with reference to the drawings.

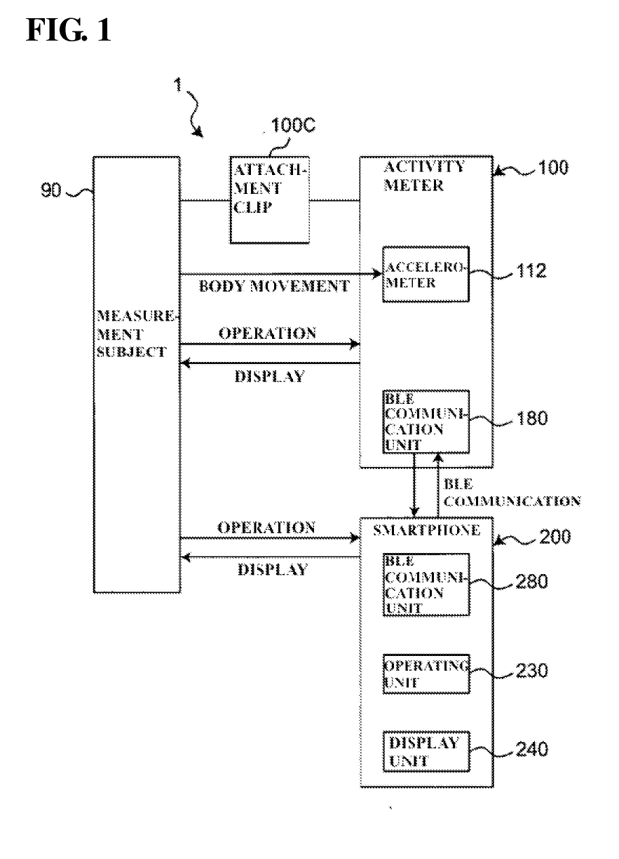

FIG. 1 illustrates a system configuration of a gait posture meter (generally indicated by reference numeral 1) according to a preferred embodiment of this invention. This gait posture meter 1 includes an activity meter 100 and a smartphone 200. In this example, the activity meter 100 and the smartphone 200 are capable of communicating with each other through BLE (Bluetooth Low Energy; Bluetooth having low energy consumption) communication.

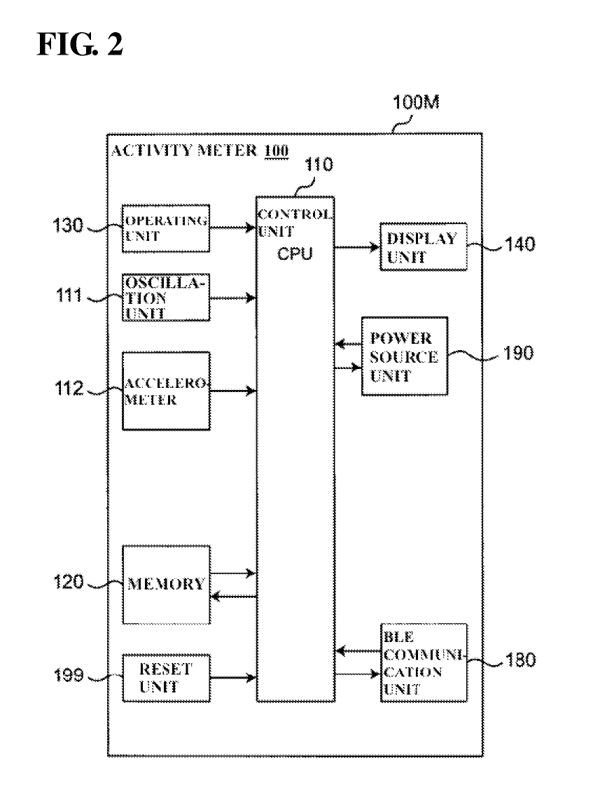

As illustrated in FIG. 2, the activity meter 100 includes a casing 100M, and a control unit 110, an oscillation unit 111, an accelerometer 112, a memory 120, an operating unit 130, a display unit 140, a BLE communication unit 180, a power source unit 190, and a reset unit 199 provided in the casing 100M.

The casing 100M is formed having a size that fits in the palm of a person's hand so that the activity meter 100 can be carried with ease.

The oscillation unit 111 includes a quartz vibrator, and emits a clock signal that serves as a reference for operational timings in the activity meter 100.

The accelerometer 112 detects accelerations in each of three axes (three directions) that the casing 100M is subjected to, and outputs those accelerations to the control unit 110.

The memory 120 includes a ROM (Read Only Memory) and a RAM (Random Access Memory). The ROM stores data of programs for controlling the activity meter 100. The RAM, meanwhile, stores configuration data for configuring various types of functions of the activity meter 100, acceleration measurement results, data of computational results, and so on.

The control unit 110 includes a CPU (Central Processing Unit) that operates based on the aforementioned clock signal, and controls the respective units of the activity meter 100 (including the memory 120, the display unit 140, and the BLE communication unit 180) based on detection signals from the accelerometer 112, in accordance with a program for controlling the activity meter 100 stored in the memory 120. The control unit 110 includes a signal processing system that at least combines an up-down axis acceleration and a front-rear axis acceleration.

The operating unit 130 is in this example constituted of button-based switches, and accepts operational inputs as appropriate, such as operations for switching the power on and off, operations for switching display details, and so on.

The display unit 140 includes a display screen that is in this example configured of an LCD (liquid-crystal display) or an organic EL (electroluminescence) display, and displays predetermined information in the display screen in accordance with signals received from the control unit 110.

The power source unit 190 is in this example a button battery, and supplies power to the various elements of the activity meter 100.

The BLE communication unit 180 communicates with the smartphone 200 in real time. For example, the BLE communication unit 180 sends information indicating measurement results and the like to the smartphone 200. The BLE communication unit 180 also receives operating instructions from the smartphone 200.

The reset unit 199 is constituted of a switch, and resets and initializes operations of the control unit 110, content stored by the memory 120, and so on.

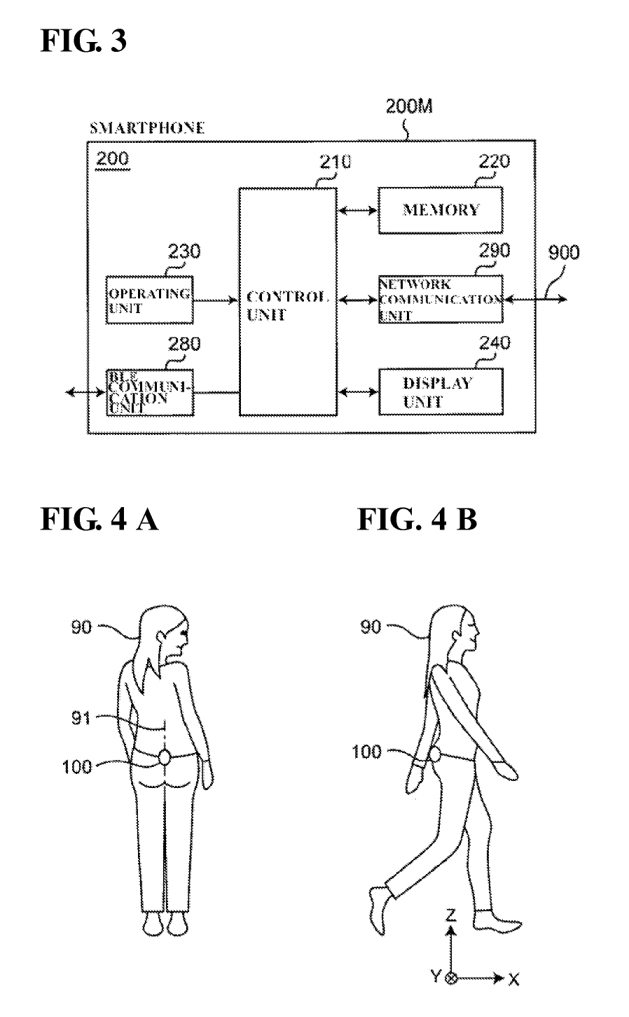

As illustrated in FIG. 3, the smartphone 200 includes a main body 200M, and a control unit 210, a memory 220, an operating unit 230, a display unit 240, a BLE communication unit 280, and a network communication unit 290 provided in the main body 200M. The smartphone 200 is a commercially-available smartphone in which application software (a computer program) for making instructions to the activity meter 100 has been installed.

The control unit 210 includes a CPU as well as auxiliary circuitry thereof, controls the various units of the smartphone 200, and executes processes in accordance with programs and data stored in the memory 220. In other words, the control unit 210 processes data inputted through the operating unit 230 and the communication units 280 and 290, and stores the processed data in the memory 220, displays the processed data in the display unit 240, outputs the processed data from the communication units 280 and 290, or the like.

The memory 220 includes a RAM used as a work area required by the control unit 210 to execute programs, and a ROM for storing basic programs to be executed by the control unit 210. A semiconductor memory (a memory card, an SSD (Solid State Drive)) or the like may be used as a storage medium in an auxiliary storage unit for complementing a storage region in the memory 220.

The operating unit 230 is in this example configured of a touch panel provided on the display unit 240. Note, however, that another hardware-based operating device such as a keyboard may be included as well.

The display unit 240 includes a display screen (constituted by, for example, an LCD or an organic EL display). The display unit 240 displays a predetermined image in the display screen under the control of the control unit 210.

The BLE communication unit 280 communicates with the activity meter 100 in real time. For example, the BLE communication unit 280 sends operating instructions to the activity meter 100. The BLE communication unit 280 also receives information expressing measurement results and the like from the activity meter 100.

The network communication unit 290 sends information from the control unit 210 to another apparatus over a network 900, and receives information sent over the network 900 from another apparatus and passes the information to the control unit 210.

As illustrated in FIG. 4A, in the case where the gait posture meter 1 is used by, for example, a measurement subject 90 serving as a user, the activity meter 100 is affixed at the waist on a rear side of the measurement subject 90, on a centerline 91 thereof, using an attachment clip 100C (indicated in FIG. 1).

In this example, relative to the measurement subject 90, a front-rear direction corresponds to the X axis, a left-right direction corresponds to the Y axis, and an up-down direction corresponds to the Z axis, as illustrated in FIG. 4B. The accelerometer 112 of the activity meter 100 outputs an X axis (front-rear axis) acceleration, a Y axis (left-right axis) acceleration, and a Z axis (up-down axis) acceleration that the casing 100M is subjected to as the measurement subject 90 walks forward.

When a measurement is to be taken using the gait posture meter 1, the measurement subject 90 turns the activity meter 100 and the smartphone 200 on. The measurement subject also launches the application software in the smartphone 200 and instructs the activity meter 100 to start measurement via the operating unit 230 and the BLE communication unit 280.

In this state, the measurement subject 90 walks straight forward by ten steps, in this example. The measurement subject 90 then instructs the activity meter 100 to perform computation and output a computational result via the operating unit 230 and the BLE communication unit 280 of the smartphone 200.

Upon doing so, the control unit 110 of the activity meter 100 operates as the computation unit, and carries out computations that will be described later. Information expressing the computational result is then sent to the smartphone 200 via the BLE communication unit 180.

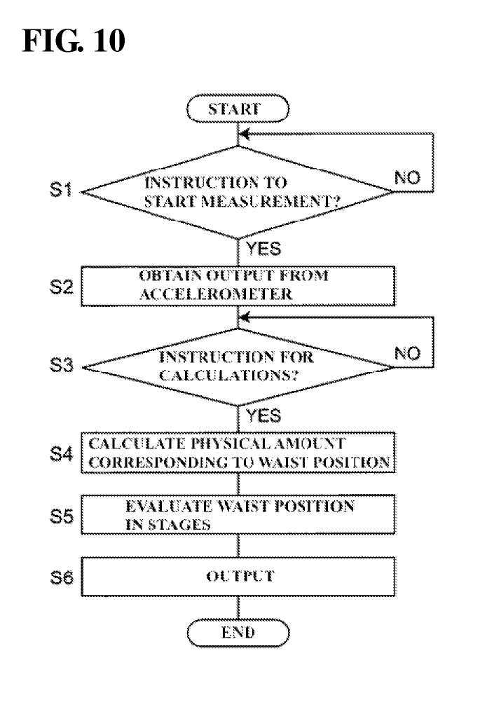

FIG. 10 illustrates a flow of operations performed by the control unit 110 of the activity meter 100. When the power is turned on, the control unit 110 of the activity meter 100 stands by for an instruction from the smartphone 200 to start measurement, as indicated in step S1. Upon receiving an instruction to start measurement from the smartphone 200 (YES in step S1), the control unit 110 obtains a three-axis output from the accelerometer 112, as indicated in step S2. The obtainment of the output from the accelerometer 112 is carried out for a predetermined period (14 seconds, for example), which serves as a period including data for ten steps in this example. The obtained data is temporarily stored in the memory 120. Next, the control unit 110 stands by for an instruction for calculations from the smartphone 200, as indicated in step S3. Upon receiving the instruction for calculations from the smartphone 200 (YES in step S3), the control unit 110 calculates physical amounts corresponding to a waist position, as indicated in step S4. Then, as indicated in step S5, the control unit 110 operates as an evaluation unit, and evaluates the waist position in stages using results of the calculation. Then, as indicated in step S6, the results of the evaluations are outputted (sent) to the smartphone 200.

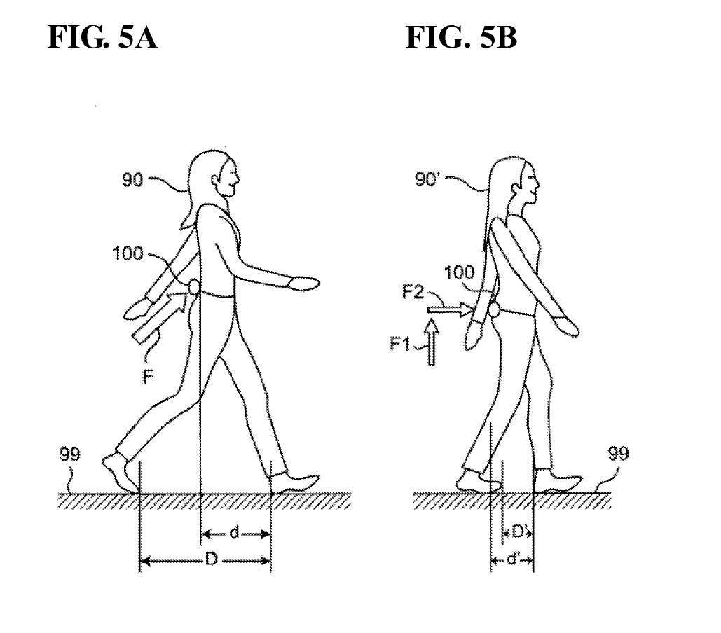

FIG. 5A illustrates the waist position relative to the rest of the body for a given measurement subject 90. Here, the waist position is expressed as follows, using a stride length (that is, a distance from the tip of the toe of the rear foot to the heel of the front foot) D and a distance d from a rear surface of the waist to the heel of the front foot, at the point in time when the heel of the front foot makes contact with a ground surface 99.

(waist position)=(distance from rear surface of waist to heel of front foot)/(stride length)=d/D (1)

The waist position of this measurement subject 90 is shifted forward, and thus the value obtained through Formula (1) (=d/D) is comparatively low.

On the other hand, FIG. 5B illustrates the waist position relative to the rest of the body for a different measurement subject 90′. The waist position of this measurement subject 90′ is shifted rearward, and thus the value obtained through Formula (1) (=d′/D′) is comparatively high.

In step S4 of FIG. 15, the stated control unit 110 calculates six physical amounts i) to vi) as described hereinafter, corresponding to the value found through Formula (1).

i) an amount expressing the magnitude of a combined vector obtained by combining a Z axis (up-down axis) acceleration and an X axis (front-rear axis) acceleration

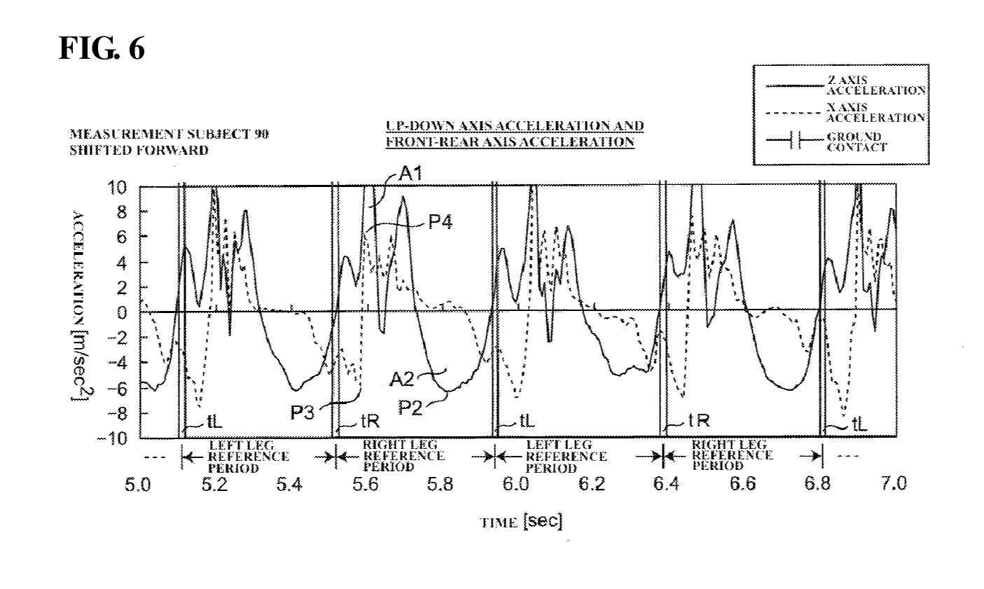

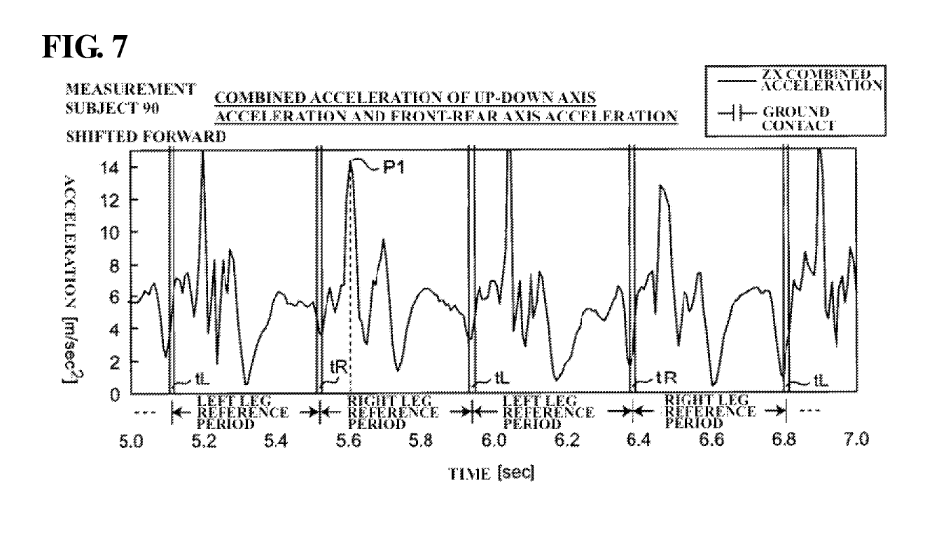

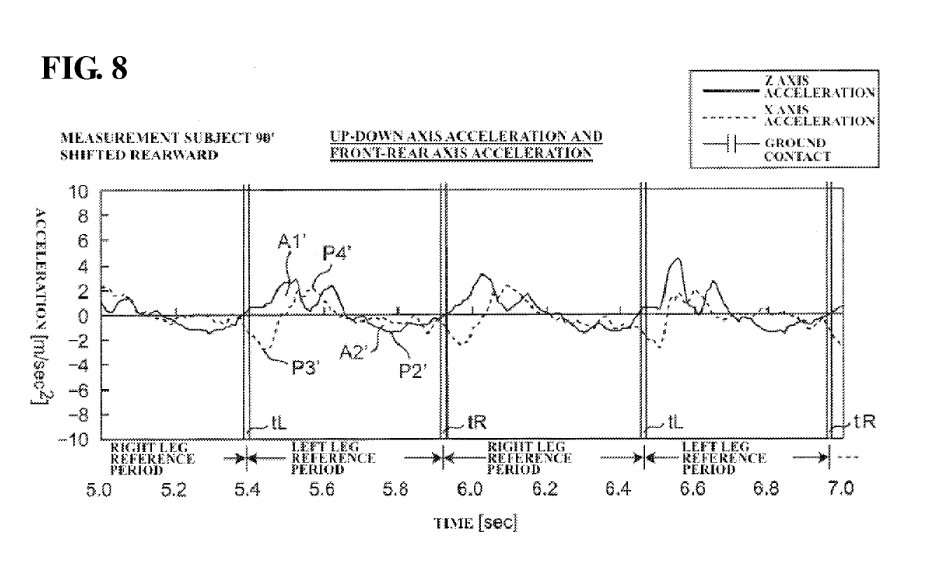

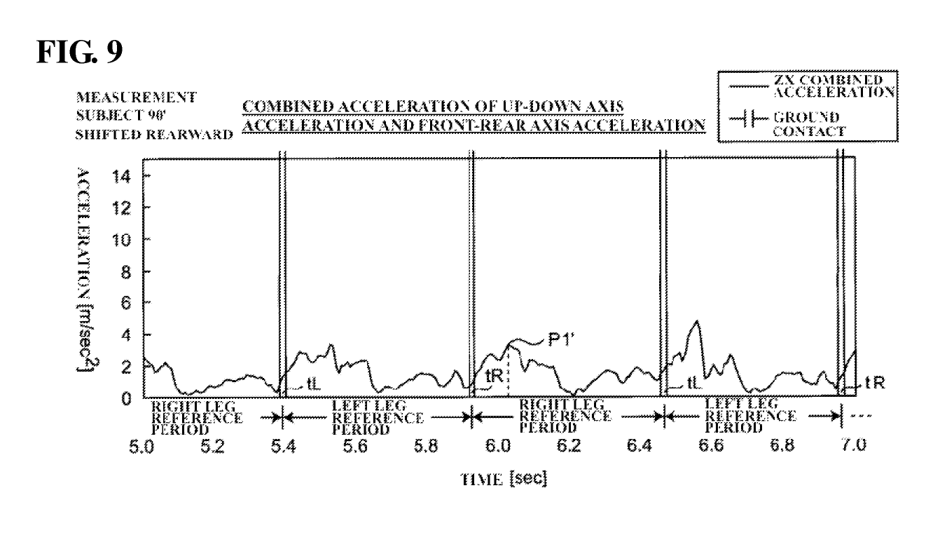

FIG. 6 is a timewise change waveform of the Z axis acceleration and the X axis acceleration for the measurement subject 90 indicated in FIG. 5A. Meanwhile, FIG. 8 is a timewise change waveform of the Z axis acceleration and the X axis acceleration for the measurement subject 90′ indicated in FIG. 5B. In FIGS. 6 and 8 (and in FIGS. 7 and 9, which will be described later), tL represents a timing at which the left heel makes contact with the ground, and tR represents a timing at which the right heel makes contact with the ground. A period spanning from when the left heel makes contact with the ground to when the right heel makes contact with the ground will be called a “left leg reference period”, and a period spanning from when the right heel makes contact with the ground to when the left heel makes contact with the ground will be called a “right leg reference period”.

In the case where the waist position is shifted forward, it is known from experience that when the rear leg, which serves as a reference, is kicked out, accelerations occur simultaneously in both the upward and forward directions. In other words, it is known from experience that accelerations occur simultaneously in both the upward and forward directions, resulting in a combined vector F, as illustrated in FIG. 5A. Conversely, in the case where the waist position is not shifted forward, it is known from experience that when the rear leg serving as a reference is kicked out, an acceleration F1 first occurs in the upward direction in order to lift the body, after which an acceleration F2 occurs in the forward direction, as indicated in FIG. 5B. In this manner, whether or not the waist position is shifted forward is related to both the up-down axis acceleration and the front-rear axis acceleration.

FIG. 7 is a timewise change waveform of a combined acceleration (a ZX combined acceleration) obtained by combining the Z axis acceleration and the X axis acceleration for the measurement subject 90 indicated in FIG. 5A. As can be seen from FIG. 7, in the case where the waist position is shifted forward, accelerations occur simultaneously in both the upward and forward directions when the rear leg serving as a reference is kicked out, and thus a peak P1 where the combined vector is high appears in both the left leg reference period and the right leg reference period. Meanwhile, FIG. 9 is a timewise change waveform of a combined acceleration (a ZX combined acceleration) obtained by combining the Z axis acceleration and the X axis acceleration for the measurement subject 90 indicated in FIG. 5B. As can be seen from FIG. 9, in the case where the waist position is not shifted forward, when the rear leg serving as a reference is kicked out, the acceleration F1 first occurs in the upward direction in order to lift the body, after which the acceleration F2 occurs in the forward direction; accordingly, a peak P1′ in the combined vector is smaller in both the left leg reference period and the right leg reference period.

Accordingly, an amount expressing the magnitude of the combined vector obtained by combining the Z axis acceleration and the X axis acceleration, and to be more specific, the value of a highest peak in both the left leg reference period and the right leg reference period, is calculated as the physical amount corresponding to the value obtained through Formula (1). Note that the magnitude of the combined vector is calculated as the square root of the sum of the square of the Z axis acceleration and the square of the X axis acceleration.

In this example, the first two steps and the last two steps of ten steps' worth of data are ignored, and the remaining six steps' worth of data is averaged to find an average value. That average value is used as a calculation result for that physical amount. Using the average value as the calculation result in this manner also applied to the remaining physical amounts ii) to vi).

ii) an amount expressing a positive-side waveform area in the timewise change waveform of the Z axis (up-down axis) acceleration

iii) an amount expressing a negative-side waveform area in the timewise change waveform of the Z axis (up-down axis) acceleration

Here, “positive-side waveform area” refers to an area obtained by integrating, with respect to time, a waveform in a time-acceleration graph such as that shown in FIGS. 6 and 8 when the acceleration has a positive value. Likewise, “negative-side waveform area” refers to an area obtained by integrating, with respect to time, a waveform in a time-acceleration graph when the acceleration has a negative value.

In the case where the waist position is shifted forward, the stride length is long and the walking speed is high; as such, it is known from experience that in both the left leg reference period and the right leg reference period in the Z axis acceleration timewise change waveform, a positive-side waveform area A1 and/or a negative-side waveform area A2 become greater, as illustrated in FIG. 6. Conversely, in the case where the waist position is not shifted forward, the stride length is short and the walking speed is low; as such, it is known from experience that in both the left leg reference period and the right leg reference period in the Z axis acceleration timewise change waveform, a positive-side waveform area A1′ and/or a negative-side waveform area A2′ become smaller, as illustrated in FIG. 8. In this manner, whether or not the waist position is shifted forward is related to the positive-side waveform area and/or the negative-side waveform area in the Z axis acceleration timewise change waveform.

Accordingly, an amount expressing the positive-side waveform area and an amount expressing the negative-side waveform area are each calculated as a physical amount corresponding to the value found through Formula (1).

iv) an amount expressing the value of a lowest valley in the negative-side waveform in the timewise change waveform of the Z axis (up-down axis) acceleration

In the case where the waist position is shifted forward, the stride length is long and the walking speed is high; as such, it is known from experience that in both the left leg reference period and the right leg reference period in the Z axis acceleration timewise change waveform, a lowest valley P2 in the negative-side waveform becomes deeper, as illustrated in FIG. 6. Conversely, in the case where the waist position is not shifted forward, the stride length is short and the walking speed is low; as such, it is known from experience that in both the left leg reference period and the right leg reference period in the Z axis acceleration timewise change waveform, a lowest valley P2′ in the negative-side waveform becomes shallower, as illustrated in FIG. 8. In this manner, whether or not the waist position is shifted forward is related to the depth of the lowest valley in the negative-side waveform in the Z axis acceleration timewise change waveform.

Accordingly, an amount expressing the depth of the lowest valley in the negative-side waveform is calculated as a physical amount corresponding to the value found through Formula (1).

Note that the magnitude of a highest peak in the positive-side waveform for the Z axis acceleration timewise change waveform corresponds less to the waist position and rather varies greatly from person to person, and is therefore difficult to use for a quantitative evaluation.

v) an amount expressing the value of a highest peak in the positive-side waveform in the timewise change waveform of the X axis (front-rear axis) acceleration

vi) an amount expressing the value of a lowest valley in the negative-side waveform in the timewise change waveform of the X axis (front-rear axis) acceleration

In the case where the waist position is shifted forward, the stride length is long and the walking speed is high; as such, it is known from experience that in both the left leg reference period and the right leg reference period in the X axis acceleration timewise change waveform, a highest peak P4 in the positive-side waveform becomes higher while a lowest valley P3 in the negative-side waveform becomes deeper, as illustrated in FIG. 6. Conversely, in the case where the waist position is not shifted forward, the stride length is short and the walking speed is low; as such, it is known from experience that in both the left leg reference period and the right leg reference period in the X axis acceleration timewise change waveform, a highest peak P4′ in the positive-side waveform becomes lower while a lowest valley P3′ in the negative-side waveform becomes shallower, as illustrated in FIG. 8. In this manner, whether or not the waist position is shifted forward is related to the height of the highest peak in the positive-side waveform and the depth of the lowest valley in the negative-side waveform in the X axis acceleration timewise change waveform.

Accordingly, an amount expressing the height of the highest peak in the positive-side waveform and an amount expressing the depth of the lowest valley in the negative-side waveform are each calculated as a physical amount corresponding to the value found through Formula (1).

In this manner, the control unit 110 calculates the stated six physical amounts i) to vi) in step S4 of FIG. 10.

Next, a method through which the control unit 110 evaluates a waist position across multiple stages in accordance with a threshold in step S5 of FIG. 10 will be described.

Note that the names of the physical amounts i) to vi) are written in a simplified manner, as “up-down/front-rear axis combined maximum value”, “up-down axis positive area”, “up-down axis negative area”, “up-down axis minimum value”, “front-rear axis maximum value”, and “front-rear axis minimum value”, respectively. The unit ism/sect for each of the physical amounts i) to vi).

In the following, thresholds (unit: m/sec²) corresponding to each of the stated six physical amounts i) to vi) and scores that take those thresholds as references will be described.

Specifically, thresholds of 5 and 10 are set for the “up-down/front-rear axis combined maximum value” physical amount i). A score of −1 point is given for a calculated up-down/front-rear axis combined maximum value of 5 or less, 0 points for more than 5 and less than 10, and +1 point for 10 or more.

Thresholds of 50 and 100 are set for the “up-down axis positive area” physical amount ii). A score of −1 point is given for a calculated up-down axis positive area of 50 or less, 0 points for more than 50 and less than 100, and +1 point for 100 or more.

Thresholds of −50 and −100 are set for the “up-down axis negative area” physical amount iii). A score of −1 point is given for a calculated up-down axis negative area of −50 or more, 0 points for less than −50 and more than −100, and +1 point for −100 or less.

Thresholds of −2.5 and −5.0 are set for the “up-down axis minimum value” physical amount iv). A score of −1 point is given for a calculated up-down axis minimum value of −2.5 or more, 0 points for less than −2.5 and more than −5.0, and +1 point for −5.0 or less.

Thresholds of 4 and 8 are set for the “front-rear axis maximum value” physical amount v). A score of −1 point is given for a calculated front-rear axis maximum value of 4 or less, 0 points for more than 4 and less than 8, and +1 point for 8 or more.

Thresholds of −3 and −6 are set for the “front-rear axis minimum value” physical amount vi). A score of −1 point is given for a calculated front-rear axis minimum value of −3 or more, 0 points for less than −3 and more than −6, and +1 point for −6 or less.

The control unit 110 calculates a total score by totaling the scores for the six physical amounts i) to vi). This total score takes on a value that changes in stages by one point each within a range from −6 points to +6 points. The waist position of that measurement subject is determined to be “shifted forward” when the total score is 0 or more. On the other hand, the waist position of that measurement subject is determined to be “shifted rearward” when the total score is −1 or less. In this manner, the control unit 110 quantitatively evaluates whether or not the waist position is shifted forward based on the total score.

Information indicating whether the waist position of that measurement subject is “shifted forward” or “shifted rearward” is outputted (sent) from the activity meter 100 to the smartphone 200 in step S6 of FIG. 10 as an evaluation result, along with the total score.

Upon receiving the information from the activity meter 100, the smartphone 200 displays the evaluation result along with the total score in the display unit 240. A message reading, for example, “your waist position is shifted forward (3 points)” is displayed in the display unit 240 of the smartphone 200. Note that a display that enables the total score to be understood intuitively, such as a bar graph expressing the total score, may be made in the display unit 240 instead of or along with the total score.

A user can quantitatively know whether or not his/her waist position is shifted forward by viewing the content displayed in the display unit 240. Such a quantitative evaluation result using a total score is easy to understand and simple for the user.

The inventors of this invention carried out experiments for verifying whether or not the quantitative evaluation results found using the gait posture meter 1 were valid, using a plurality of measurement subjects.

Specifically, for each measurement subject, a photograph was taken when the heel of the front foot made contact with the ground surface 99 when walking, and the waist position (% value) was found through Formula (1) based on the photographic image. In addition, a quantitative evaluation result (the aforementioned total score) was found for each of the measurement subjects using the gait posture meter 1. A correlation between the waist position (% value) found through Formula (1) based on the photographic image and the quantitative evaluation result (the aforementioned total score) from the gait posture meter 1 was then examined.

As a result, results indicating a correlation coefficient R of 0.81 and an error (standard deviation) SD of 1.7 were obtained for a given measurement subject group made up of 31 measurement subjects (76 pieces of data). Furthermore, results indicating a correlation coefficient R of 0.68 and an error (standard deviation) SD of 1.9 were obtained for a different measurement subject group made up of 25 measurement subjects (65 pieces of data).

Accordingly, the quantitative evaluation results from the gait posture meter 1 could generally be verified as valid.

As described thus far, according to the gait posture meter 1, a measurement subject's waist position while walking can be quantitatively and correctly evaluated in accordance with the aforementioned physical amounts i) to vi). Furthermore, the gait posture meter 1 carries out the evaluation based on the output of the accelerometer 112, and thus the evaluation can be made easily, without requiring large-scale equipment such as is used for motion capture or the like.

Although the accelerometer 112 is affixed on the centerline of the measurement subject's waist in the aforementioned preferred embodiment, the invention is not limited thereto. Assuming that the accelerometer 112 is affixed in a given direction relative to the measurement subject, the control unit 110 may constitute a signal processing system that combines and extracts the up-down axis acceleration, the front-rear axis acceleration, and so on based on three mutually-perpendicular directional components relative to the accelerometer 112 as outputted by the accelerometer 112. In this case, the control unit 110 operates as the computation unit, and quantitatively calculates physical amounts corresponding to the measurement subject's waist position while walking using one or both of the up-down axis acceleration timewise change waveform and the front-rear axis acceleration timewise change waveform outputted by the stated signal processing system. Accordingly, the measurement subject's waist position while walking can be quantitatively evaluated in accordance with those physical amounts. In such a case, the accelerometer 112 (and/or the casing 100M in which the accelerometer 112 is mounted) can be affixed in any desired direction, such as in a pocket of the measurement subject's clothes, without restricting the orientation of the accelerometer 112 relative to the measurement subject. This improves the usability for the user.

Although the six physical amounts i) to vi) are calculated in the aforementioned preferred embodiment, the invention is not limited thereto. For example, rather than calculating all six physical amounts i) to vi), only some of the physical amounts, such as i), may be calculated, and the measurement subject's waist position while walking may be quantitatively evaluated using only that physical amount i).

Although the activity meter 100 and the smartphone 200 communicate with each other through BLE communication in the aforementioned preferred embodiment, the invention is not limited thereto. For example, the activity meter 100 and the smartphone 200 may communicate through NFC (Near Field Communication) when the smartphone 200 and the activity meter 100 are near each other.

In addition, although the gait posture meter according to the present invention is described as being configured as a system including the activity meter 100 and the smartphone 200 in the aforementioned preferred embodiment, the invention is not limited thereto.

For example, the gait posture meter according to the present invention may be constituted by the smartphone 200 only. Such a case assumes that the smartphone 200 includes an accelerometer. In addition, a program that causes the control unit 210 to quantitatively evaluate whether or not the gait posture of a person is a correct posture, and more specifically, a program that quantitatively evaluates whether or not the waist position while walking is shifted forward, is installed in the memory 220 of the smartphone 200. Through this, the gait posture meter according to the present invention can be configured as a small-sized, compact unit.

This program can be recorded onto a recording medium such as a CD, a DVD, a flash memory, or the like as application software. By installing the application software recorded onto the recording medium in what is substantially a computer device, such as a smartphone, a personal computer, a PDA (personal digital assistant), or the like, that computer device can be caused to execute a method for quantitatively evaluating whether or not the gait posture of a person is a correct posture.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A gait posture meter that evaluates a gait posture of a measurement subject, the meter comprising:
an accelerometer affixed to a centerline of a measurement subject's waist area;
a computation unit that quantitatively calculates a physical amount corresponding to a waist position of the measurement subject while walking in a front-rear direction relative to the rest of the body using one or both of a timewise change waveform of an up-down axis acceleration and a timewise change waveform of a front-rear axis acceleration outputted by the accelerometer; and
an evaluation unit that evaluates whether or not the waist position while walking is shifted forward in the front-rear direction relative to the rest of the body based on the physical amount.

2. The gait posture meter according to claim 1,
wherein the computation unit includes a signal processing system that combines the up-down axis acceleration and the front-rear axis acceleration; and
the physical amount includes an amount regarding a combined vector obtained by combining the up-down axis acceleration and the front-rear axis acceleration.

3. The gait posture meter according to claim 2,
wherein the amount regarding the combined vector is a magnitude of the combined vector.

4. The gait posture meter according to claim 1,
wherein the physical amount includes an amount expressing a positive-side waveform area and/or a negative-side waveform area in the timewise change waveform of the up-down axis acceleration, in each of a left leg reference period spanning from when a left heel makes contact with the ground to when a right heel makes contact with the ground and a right leg reference period spanning from when the right heel makes contact with the ground to when the left heel makes contact with the ground.

5. The gait posture meter according to claim 1,
wherein the physical amount includes an amount expressing a value of a lowest valley in the negative-side waveform in the timewise change waveform of the up-down axis acceleration, in each of the left leg reference period spanning from when the left heel makes contact with the ground to when the right heel makes contact with the ground and the right leg reference period spanning from when the right heel makes contact with the ground to when the left heel makes contact with the ground.

6. The gait posture meter according to claim 1,
wherein the physical amount includes an amount expressing a value of a highest peak in a positive-side waveform and/or a value of a lowest valley in a negative-side waveform in the timewise change waveform of the front-rear axis acceleration, in each of the left leg reference period spanning from when the left heel makes contact with the ground to when the right heel makes contact with the ground and the right leg reference period spanning from when the right heel makes contact with the ground to when the left heel makes contact with the ground.

7. The gait posture meter according to claim 1,
wherein the evaluation unit sets a threshold for the physical amount and evaluates the waist position while walking in the front-rear direction relative to the rest of the body across multiple stages in accordance with the threshold.

8. A non-transitory computer readable medium including a computer program for causing a computer to execute a method for evaluating a gait posture of a measurement subject, the method comprising:
a step of obtaining an output of an accelerometer that is affixed to a centerline of the measurement subject's waist area;
a step of quantitatively calculating a physical amount corresponding to a waist position of the measurement subject while walking in a front-rear direction relative to the rest of the body using one or both of a timewise change waveform of a front-rear axis acceleration and a timewise change waveform of an up-down axis acceleration outputted by the accelerometer; and
a step of evaluating whether or not the waist position while walking is shifted forward in the front-rear direction relative to the rest of the body based on the physical amount.
